(19) United States
(12) Patent Application Publication
(10) Pub. No.: US 2025/0041536 A1
(43) Pub. Date: Feb. 6, 2025

(54) DRUG DELIVERY ARRANGEMENT COMPRISING A TISSUE RECEIVING ELEMENT

(71) Applicant: Sanofi, PARIS (FR)

(72) Inventors: Max Davis, Melbourn, Hertfordshire (GB); Thomas Mark Kemp, Melbourn, Hertfordshire (GB); Tom Lever, Melbourn, Hertfordshire (GB); Max Wehebrink, Melbourn, Hertfordshire (GB); Robbie Wilson, Melbourn, Hertfordshire (GB)

(21) Appl. No.: 18/719,711

(22) PCT Filed: Dec. 13, 2022
(86) PCT No.: PCT/EP2022/085641

§ 371 Date: Jun. 13, 2024

(30) Foreign Application Priority Data
Dec. 15, 2021 (EP) 21315276.2

(51) Int. Cl. A61M 5/46

(52) U.S. Cl. CPC A61M 5/46

(57) ABSTRACT

A drug delivery arrangement (1) comprising a drug delivery device (2), having a housing (3), and a needle, and a tissue receiving element (6), having a needle passage opening (7) and a tissue receiving portion (8), wherein the tissue receiving element is configured to interact with the skin (9) in order to lift tissue into the tissue receiving portion to reduce the risk of injury due to deep punctures.

### BACKGROUND

To both reduce the risk of injury to a patient when delivering a drug and to ensure effective delivery of the drug into the patient's body, the depth of penetration of the needle into the patient's body is important. Commercially available drug delivery devices, especially auto-injectors, often have a mechanism that only has a fixed needle penetration depth. However, this has disadvantages. For some patients, a fixed needle penetration depth may be either too deep or not deep enough to ensure safe delivery of the drug. This problem arises particularly when the drug delivery device should be used for children. Children, in general, have less tissue available, which can be penetrated by a needle.

### SUMMARY

It is an object of the present disclosure to facilitate improvements associated with drug delivery arrangements, particularly with respect to user safety of the injection depth.

This object is achieved by subject-matter disclosed herein, for example by the subject-matter defined in the appended independent claim. Advantageous refinements and developments are subject to dependent claims and/or set forth in the description below.

One aspect of the present disclosure relates to a drug delivery arrangement. The drug delivery arrangement comprises a drug delivery device having a housing, the housing being provided to receive a drug container and a needle.

The needle is expediently configured to pierce a skin of a user. Through the needle, the medicament may be administered to the user, e.g. into the user's tissue. In one embodiment, the needle, e.g. when received in the housing, is axially fixed relative to the housing. That is to say, axial movement of the needle relative to the housing may be prevented, preferably in the distal direction and/or in the proximal direction. The needle may be received in the housing of the drug delivery device. We note however, that the presently disclosed concepts do also apply to devices comprising or provided to retain a movable needle, which is configured to move relative to the housing, e.g. driven by a spring, for piercing the skin and/or for the drug delivery operation.

In one embodiment, the needle may be in or may be brought into fluid communication with an interior of the drug container. The needle may be integrated into the drug container. The drug, e.g. a liquid medicament, is expediently arranged in the interior of the container. The drug container may be a syringe, e.g. a syringe with a preinstalled needle, such as a staked needle. Alternatively, the drug container may be a cartridge, which may have to be brought into fluid communication with a separate needle unit, e.g. by piercing a cartridge septum with the needle of the needle unit.

In addition, the drug delivery arrangement comprises a tissue receiving element, having a needle passage opening and a tissue receiving portion. The tissue receiving element is arranged to contact the skin of the patient during use of the drug delivery arrangement, wherein the tissue receiving portion extends in the distal direction away from the needle passage opening. The drug delivery arrangement is configured such that the needle passage opening and the needle are movable relative to one another from a first position, in which the needle is arrange outside of the tissue receiving portion, into a second position, in which the needle is arranged within the tissue receiving portion, wherein the needle enters the tissue receiving portion during the relative movement from the first position into the second position to penetrate tissue arranged in the tissue receiving portion. The tissue receiving element is configured to interact with the skin in order to fill the tissue receiving portion with tissue.

The tissue receiving portion of the tissue receiving element allows the penetration area on the skin to be adjusted to the penetration depth of the needle. Thus, the drug delivery assembly reduces the risk of injuring the patient when delivering the drug and ensures reliable delivery of the drug, especially when the patient is a patient with thinner tissue, such as a child. Adjustment of the skin area into which the needle enters can be achieved by manipulating the skin in a way that causes the skin to move towards the needle passage opening. In this way, the tissue thickness is increased in the area where the needle penetrates the skin. Moreover, the drug delivery arrangement being configured such that the needle passage opening and the needle are movable relative to one another can achieve that the needle is not inserted before the tissue thickness available for penetration in the tissue receiving portion has been manipulated. This also leads to an increased application safety of the drug delivery arrangement.

In one embodiment, the tissue receiving portion may taper continuously as seen from its distal end towards the needle passage opening. In other words, a cross-section of the tissue receiving portion may reduce from its distal end in a proximal direction. Alternatively, or additionally, the tissue receiving portion may transition to the needle passage opening. For example, a proximal end of the tissue receiving portion may transition to a distal end of the needle passage opening.

In one embodiment, the tissue receiving element is part of the drug delivery device. However, the tissue receiving element may also be attachable to the drug delivery device.

In one embodiment, the needle passage opening is the distal end portion of a guide channel or track within the housing or a needle shroud through which, and in which, the needle can move.

The term “distal” or “distal end” designates that end of the drug delivery device or a component thereof, which is or is to be arranged closest to a dispensing end of the drug delivery device. The term “proximal end” designates that end of the device or a component thereof, which is or is to be arranged furthest away from the dispensing end of the device. The distal end and the proximal end are spaced apart from one another in the direction of an axis. The axis may be the longitudinal axis of the drug delivery arrangement or elements thereof.

In one embodiment, the drug delivery arrangement is configured such that the needle tip is not able to touch the patient's skin when the skin is bulged in the tissue receiving portion and the needle is in the first position. In this way, unintentional injuries caused by the needle can be prevented.

In one embodiment, the tissue receiving portion has a larger diameter in a distal end region, this end region being the region furthest from the proximal end of the tissue receiving portion, than the needle passage opening. The tissue receiving portion may taper in the direction of the needle passage opening. This shape supports the deformation of the skin in the area of the needle entry point.

In one embodiment, the diameter in the distal end region of the tissue receiving portion can be greater than 18-20 mm, for example 30 mm.

In one embodiment, the tissue receiving element comprises a wall extending in the distal direction as seen from the needle passage opening, wherein the tissue receiving portion is laterally delimited by an inner surface of the wall. The wall serves as a contact surface for the skin, allowing the skin to deform when the drug delivery arrangement is pressed against the skin. By designing the shape and size of the wall, it is possible to specifically influence the way in which the skin is manipulated during the delivery of the medication.

In one embodiment, the tissue receiving element comprises arms defining the wall that are movable relative to each other. Moreover the arms may be designed to be elastically flexible. The movability of the arms allows the arms to flex slightly when the drug delivery arrangement is pressed against the skin so as not to cause pain to the patient.

In one embodiment, the arms are diametrically opposed in the radial direction of the drug delivery arrangement. The tissue receiving element can also comprise more than two arms, such as 3, 4, 5, 6, 7 or 8, each equally spaced along the circumference of the tissue receiving portion. Further, in one embodiment, the number of arms is increased such that the arms are arranged immediately adjacent to each other so that the arms form a bowl shape.

In one embodiment, the cross section of the wall is curved. Viewed from the distal end in axial direction to the proximal end, the wall can be concave. This means that the arms are curved around the tissue receiving portion. The wall may have a U-shape cross section. Such a shape allows the skin to rest evenly on the wall so that a secure hold of the device on the skin is ensured. In particular, however, these shapes cause the skin in the area of the puncture site to bulge upwards towards the needle passage opening.

In one embodiment, the drug delivery arrangement comprises a needle shroud. The needle shroud may be provided to cover the needle. The needle shroud may be provided to cover the needle before the needle pierces the skin and/or after the needle has been removed from the skin, e.g. after completion of the drug delivery operation. Before the drug delivery operation is commenced, the needle shroud may protrude distally from the housing, e.g. to cover the tip of the needle (such as by axially extending beyond the tip of needle in the distal direction). For the drug delivery operation, the needle shroud may be displaced proximally relative to the housing. After completion of the drug delivery operation, the needle shroud may be moved distally relative to the housing, e.g. to cover the tip of needle. The drug delivery device may comprise a shroud spring. The shroud spring may be operatively coupleable to or coupled to the needle shroud in order to move the needle shroud, e.g. into the distal direction relative to the housing. The force of the shroud spring may have to be overcome in order to move the needle shroud in the proximal direction. In a final position, e.g. after the drug delivery operation has been completed and the device or the arrangement has been removed from the skin, the needle shroud may be locked against proximal movement with respect to the housing, such as by a locking mechanism.

In one embodiment, the needle shroud is an activation member. The activation member is a member which may have to be moved relative to the housing in order to enable triggering of the drug delivery operation or to trigger the drug delivery operation. Enable triggering of the drug delivery operation may comprise that in addition to movement of the activation member another member such as a trigger member has to be actuated, e.g. a trigger button has to be pressed.

In one embodiment, the needle shroud comprises a needle shroud body wherein the tissue receiving element is arranged at the distal end of the needle shroud body or attachable to the distal end of the needle shroud body. The tissue receiving element may be detachably connected to the needle shroud body. In other words, the tissue receiving element may be preinstalled to the needle shroud and may be removable from the needle shroud. The tissue receiving element may be connected or connectable to the needle shroud by a thread or by a snap lock. It is therefore possible to combine the needle shroud with different shapes and dimensions of tissue receiving elements. Alternatively, the tissue receiving element may be an integral part of the needle shroud body, i.e. the needle shroud and the tissue receiving element are in this case one piece.

In one embodiment, tissue receiving element may be detachably connected to the housing. In other words, the tissue receiving element may be preinstalled to a distal end portion of the housing and may be removable from the housing for example by a thread or by a snap lock. The tissue receiving element can be removed from the housing to connect different shapes and dimensions of various tissue receiving elements to the housing. Alternatively, the tissue receiving element may be an integral part of the housing, i.e. housing and tissue receiving element are in this case one piece. In this embodiment, it is possible that the drug delivery arrangement does not have a needle shroud and that the tissue receiving element is attached to the housing, wherein the needle is axially movable relative to the housing.

The drug delivery arrangement may be configured such that, when the tissue receiving element has been removed or disconnected from the drug delivery device, it cannot be reconnected, e.g. by providing a connection feature which breaks when the tissue receiving element is removed from the drug delivery device.

In one embodiment, the tissue receiving element is disconnected from the drug delivery device, e.g. when the drug delivery arrangement is provided by the manufacturer. Preferably, the user can decide whether to connect the tissue receiving element to the device member or not. Once the tissue receiving element has been connected to the drug delivery device, the connection may be releasable or irreleasable (non-releasable).

In one embodiment, the drug delivery arrangement further comprises an energy storage unit. The energy storage unit may be a drive spring or another type of energy source such as a gas reservoir. The drug delivery device is configured to perform a drug delivery operation, e.g. using energy obtainable from the energy storage unit. The energy storage unit may be configured to provide energy for the drug delivery operation of the drug delivery device. The energy may be used to drive a drive member, e.g. a plunger rod, of the drug delivery device in order to dispense drug from the drug container. For the drug delivery operation, the drive member may be displaced in a distal direction relative to the housing by the energy provided by the energy storage unit.

In one embodiment, the drug delivery device is an autoinjector. In an autoinjector the energy for the drug delivery operation may be prestored in the energy storage unit. That is to say, the user does not have to provide the energy for the drug delivery operation, e.g. when preparing the device for use. Rather, this energy may be preloaded into the system by the manufacturer. For example, a drive spring may be pre-stressed or pre-biased to provide the energy for the drug delivery operation.

In one embodiment, the drug delivery arrangement comprises a plunger release mechanism configured to have a first state and a second state, wherein the stored energy of the energy storage unit is released when the plunger release mechanism is in the second state.

In one embodiment, the energy storage unit can contain a preloaded spring. The spring may be a coil spring, for example.

In one embodiment, the plunger release mechanism is switched from the first state to the second state after the needle is moved relative to the needle passage opening from the first position to the second position.

In one embodiment, the needle shroud is axially movable relative to the housing from a first needle shroud position to a second needle shroud position, and wherein the plunger release mechanism is configured such that the plunger release mechanism is switched from the first state to the second state after the needle shroud is moved from the first needle shroud position to the second needle shroud position.

In one embodiment, the needle is configured such that after the energy storage unit has released the energy, the needle moves from the second position to the first position.

In one embodiment, the needle shroud is configured such that after the energy storage unit has released the energy, the needle shroud moves from the second needle shroud position to the first needle shroud position.

In one embodiment, the needle is not movable relative to the housing, i.e. the needle is fixed relative to the housing.

In one embodiment, the needle is exclusively movable from the first position to the second position if the needle shroud is in the second needle shroud position.

In one embodiment, the drug delivery device assembly comprises a trigger button, wherein the stored energy of the energy storage unit is released when the plunger release mechanism is in the second state and after the trigger button has been operated by the user.

In one embodiment, the needle is movable relative to the housing. Furthermore, the needle may exclusively move relative to the housing in the second state.

In one embodiment, the drug delivery arrangement comprises a cap, wherein the cap comprises recesses in the radial direction which are suitable for receiving a portion of the tissue receiving element. The recesses may be suitable for receiving the inner surface of the wall of the tissue receiving element.

In one embodiment, the cap may cover and/or receive the tissue receiving element.

In one embodiment, the cap is configured such that the outer surface of the wall, that is, the surface of the wall facing away from the tissue receiving portion, is not covered by the cap when the cap is mated to the tissue receiving element.

In one embodiment, the cap may cover a distal end, e.g. the needle end, of the drug delivery device or the arrangement and be removable or detachable from the drug delivery device or the arrangement.

In one embodiment, the needle passage opening of the drug delivery device or the arrangement may become exposed, when the cap is removed.

In one embodiment, the cap comprises a needle shield remover. The needle shield remover is, preferably, axially locked to a needle shield, which may cover the needle, e.g. when the cap is in place on the drug delivery device or the drug delivery arrangement. Hence, the needle shield may be removed together with the cap from the drug delivery device, e.g. due to an interaction between the needle shield remover and the needle shield. The needle shield may cover the distal end of the needle (the needle tip) when connected to the needle.

We note that features described above and below in conjunction with different embodiments or aspects can be combined with one another, even if such a combination is not explicitly disclosed herein above or below. Further features, advantages and expediencies of the disclosure and, particularly, of the proposed concepts will become apparent from the following description of the exemplary embodiments in conjunction with the drawings.

### BRIEF DESCRIPTION OF THE DRAWINGS

FIG. 1 illustrates an isometric view of an exemplary embodiment of the drug delivery arrangement according to the present invention.

FIG. 2 illustrates a sectional side view of a portion of the embodiment of FIG. 1 prior to a dispensing operation.

FIG. 3 illustrates another sectional side view of the portion of the embodiment of FIG. 1 during the dispensing operation.

FIG. 4 illustrates an isometric view of a tissue receiving element and a portion of a drug delivery device according to the present invention.

FIG. 5 illustrates an isometric view of a cap according to the invention joined to the tissue receiving element.

FIG. 6 illustrates a sectional side view of the portion of the embodiment of FIG. 1 with the cap mounted thereon.

FIG. 7 illustrates another sectional side view of the part of the embodiment of FIG. 1 during cap removal.

### DESCRIPTION OF EXEMPLARY EMBODIMENTS

Identical elements, elements of the same kind and identically or similarly acting elements may be provided with the same reference numerals in the drawings.

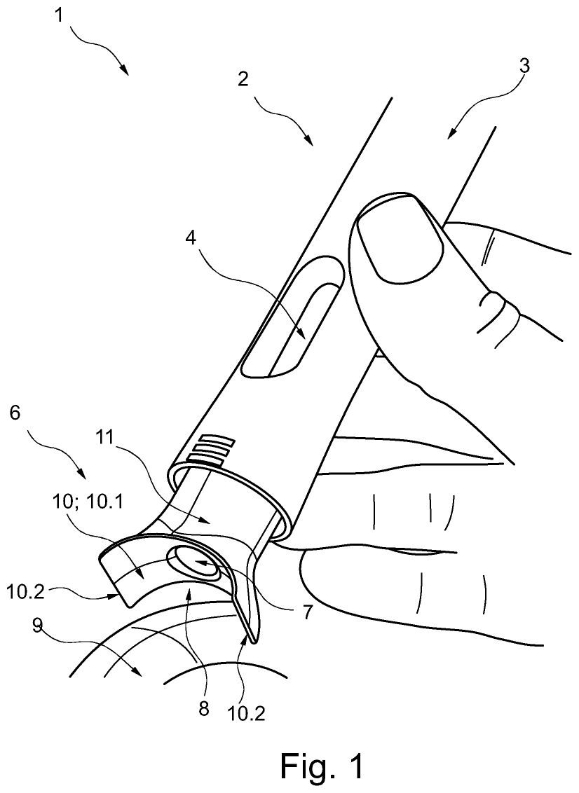

FIGS. 1 to 7 illustrate the same embodiment of a drug delivery arrangement 1 according to the present invention.

The drug delivery arrangement 1 comprises a drug delivery device 2. The drug delivery device 2 is very similar to the device disclosed in WO 2015/004052 A1, the entire disclosure content of which is incorporated herein by reference for all purposes, especially with respect to the design of the drive mechanism or “plunger release mechanism” as it is termed therein. The drug delivery device 2 having a housing 3, a drug container 4 and a needle 5 (needle 5 not shown in FIG. 1). The housing 3 being provided to receive the drug container 4, wherein the drug container 4 in FIG. 1 is visible through an opening or window in the housing 3. The housing 3 expediently covers the majority of the length of the drug delivery device 2, e.g. 60% or more or 70% or more or 80% or more of the entire length of the drug delivery device 2.

As shown in FIG. 1, the drug delivery arrangement 1 additionally comprises a tissue receiving element 6. The tissue receiving element 6 comprises a needle passage opening 7 and a tissue receiving portion 8. The tissue receiving element 6 is adapted to contact and bear against a skin 9 of a patient. Hence, the distal surface of the tissue receiving element 6 provides a bearing surface. The bearing surface may extend around a needle passage opening 7. The tissue receiving portion 8 extends in the distal direction away from the needle passage opening 7. The tissue receiving portion 8 is delimited by an inner surface 10.1 of a wall 10. The wall 10 is arranged at the distal direction of the needle passage opening 7, wherein the inner surface 10.1 is the bearing surface.

The housing 3 is provided to retain and/or retains the drug container 4. Medicament, e.g. liquid medicament, is arranged in the drug container 4. The housing 3 is provided to retain and/or retains the needle 5. In other words, a needle 5 may be arranged or arrangeable in the housing 3. The needle 5 can be an integral part of the drug container 4, e.g. (permanently or releasably) connected to a drug container body, or separate from the drug container. In the first case, the drug container 4 may be a syringe. In the second case, the drug container 4 may be a cartridge. In case a cartridge is used as drug container 4, initially, the drug container and the needle 5 can be fluidly disconnected and fluid communication between the drug container interior and the needle is only established during operation of the drug delivery device 2.

In addition a drive mechanism provided to drive a drug delivery operation is expediently provided in the housing 3. The drive mechanism comprises a plunger rod (not explicitly shown). In the shown embodiment the drug delivery device 2 is an auto-injector.

As depicted in FIG. 1, the drug delivery arrangement 1 comprises a needle shroud 11, wherein the needle shroud 11 protrudes distally from the housing 3. The needle shroud 11 is movable in a proximal direction relative to the housing 3. The needle shroud 11 may also be movable in a distal direction relative to the housing 3. The needle shroud 11 has a needle shroud body, wherein the tissue receiving element 6 is arranged at the distal end of the needle shroud body.

The needle shroud 11 is movable relative to the housing 3 from an initial position or first needle shroud position C to a second needle shroud position D. The second needle shroud position D may be a trigger position.

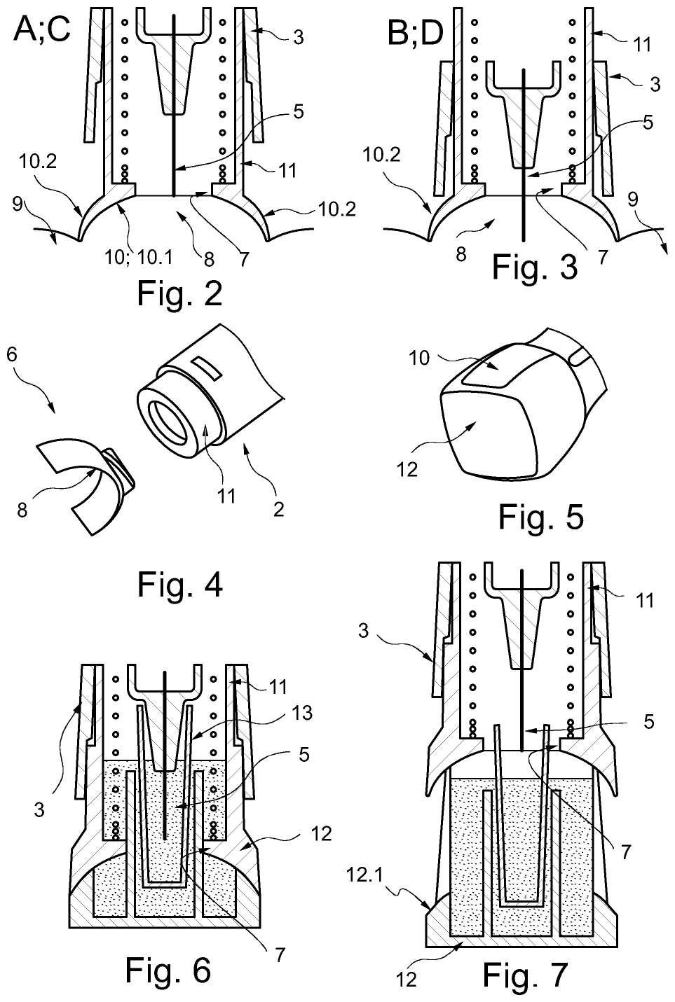

As depicted in FIG. 4, the tissue receiving element 6, having the tissue receiving portion 8 and the wall 10, is detachably attached to the drug delivery device 2, for example by a thread or by a snap lock. The wall 10 comprises two arms 10.2. The arms 10.2 are diametrically opposed in the radial direction of the drug delivery arrangement 1. As shown particular in FIGS. 2, 3, 6 and 7 the wall has a U-shape cross section. The cross section of the wall 10 can also be curved or concave. Therefore, the tissue receiving portion 8 has a larger diameter in a distal end region than the needle passage opening 7. The tissue receiving portion 8 may taper continuously as seen from its distal end towards the needle passage opening.

FIG. 2 illustrates the needle 5 in a first position A and FIG. 3 illustrates the needle 5 in a second position B relative to the needle passage opening 7. FIG. 2 also illustrates the needle shroud 11 in the first needle shroud position C while FIG. 3 illustrates the needle shroud 11 in the second needle shroud position D relative to the housing 3. In the first position A the needle 5 is arrange outside of the tissue receiving portion 8, wherein in position A the needle 5 cannot pierce the skin 9. In the second position B the needle 5 is arranged within the tissue receiving portion 8. In the second position B, the needle 5 has pierced the skin 9 and the distal needle tip is arranged in the tissue below the skin surface so that a drug can be delivered from the needle 5 into the tissue. In the shown embodiment of FIGS. 1 to 7 the drug delivery arrangement 1 is configured such that the needle passage opening 7 and the needle 5 are movable relative to one another from a first position A into the second position B. Furthermore, the needle shroud 11 is axially movable relative to the housing 3 in the proximal direction from the first needle shroud position C to the second needle shroud position D.

The drug delivery arrangement 1 comprises a cap 12 for covering the needle passage opening 7. The cap is shown in FIGS. 5 to 7. The cap 12 is arranged at the distal end of the drug delivery arrangement 1. The cap 12 is detachably connected to the remainder of the arrangement, e.g. to the tissue receiving element 6. The cap 12 can be additionally or alternatively detachably connected to the housing 3 and/or another component or member of the drug delivery device 2. The cap 12 covers a distal end of the needle shroud 11 and the needle passage opening 7 through which the needle 5, e.g. the distal needle tip, passes to pierce the skin 9 from the interior of drug delivery device 2 during or for the drug delivery operation.

The cap 12 can comprise a needle shield remover, i.e. a gripper, which engages a needle shield 13 which covers the needle 5 such that the needle shield 13 is removed from the needle 5 together with the cap 12, e.g. when the cap is detached or disconnected from the drug delivery arrangement 1. As can be seen in FIG. 7, the diameter of the needle passage opening 7 is designed in such a way that the needle shield remover together with the needle shield 13 can be moved through the needle passage opening 7 in the axial direction when removing the cap 12. As depicted in FIG. 5, the cap 12 comprises recesses 12.1 in the radial direction which are suitable for receiving a portion of the tissue receiving element 6. The recesses 12.1 are diametrically opposed in the radial direction of the drug delivery arrangement 1. Preferably, as depicted in FIG. 6, the dimensions of the recesses 12.1 are such that the inner surface of the wall 10.1 comes to rest against the recesses 12.1 when the cap 12 is fitted onto the tissue receiving element 6.

In the embodiment shown in FIGS. 1 to 7, the use of the drug delivery arrangement 1 according to the invention begins with the removal of the cap 12 from the tissue receiving element 6. FIG. 6 shows the drug delivery arrangement 1 with the attached cap 12. After the cap 12 has been removed, the needle passage opening 7 and the inner surface 10.1 of the wall 10 can be placed on the patient's skin 9. As can be seen in FIGS. 2 and 3, the shape of the wall 10 causes the area of skin located between the two arms 10.2 of the wall 10 to bulge upwards in a proximal direction when force is applied to the two arms 10.2 in a distal axial direction. The arms 10.2 are rigid, i.e. they can react a radial and/or axial force acting on it. In the embodiment shown, the arms 10.2 may be slightly elastic in the radial direction so that pressing the arms 10.2 onto the patient's skin does not cause pain. Furthermore, the arms pressed into the skin surface improve the grip of the drug delivery arrangement 1 on the skin 9, so that slipping of the drug delivery arrangement 1 during a delivery process is counteracted. After the drug delivery arrangement 1 has been placed on the skin 9, the drug delivery arrangement 1 is in a state as shown in FIG. 2. In this state, the needle 5 is in the first position A and the needle shroud 11 is in the first needle shroud position C.

The drug delivery arrangement 1 according to the embodiment comprises an energy storage unit, e.g. a drive spring, such as a compression spring, (not shown). The energy storage unit is arranged to drive a plunger rod in a distal direction relative to the drug container 4 during the drug delivery operation. During this movement, a stopper, which is movably retained in the medicament container and may seal the drug container 4 proximally, can be displaced towards an outlet of the drug container 4 to dispense the drug or medicament retained within the drug container 4 through the outlet. The outlet may be formed or defined by the needle 5. Other potential drive energy sources different from a spring comprise an electrical power cell or battery for driving the plunger rod by a motor or a reservoir suitable to provide gas pressure, where the gas pressure can be used to drive the drug delivery operation. The drug delivery device 2 is an autoinjector. The energy for driving the drug delivery operation in an autoinjector may be provided by components integral to the drug delivery device 2 and does not have to be loaded into the device by the user during the operation of the device as is the case in many spring driven pen-type variable dose injectors, where, usually, the energy is loaded into the spring by the user during a dose setting procedure. The drug delivery device 2 expediently is a single shot device, i.e. it is provided to dispense only one dose. The drug delivery device 2 may be disposable drug delivery device, that is to say a device which is disposed of after its use. The device may be a pen-type device. The medicament container 4 and/or the needle 5 can be axially secured within the drug delivery device 2, e.g. within the housing 3, or can be movable relative to the housing 3, e.g. for piercing the skin. In the first case, the user may have to perform the movement for piercing the skin 9 with the needle 5. In the second case, piercing of the needle 5 may be driven by a needle insertion mechanism of the drug delivery device 2.

Furthermore the drug delivery arrangement 1 comprises a plunger release mechanism (not shown) configured to have a first state and a second state. In the embodiment shown, the plunger release mechanism prevents the release of energy from the energy storage unit in the first state and allows the release of energy from the energy storage unit in the second state. In FIG. 2, the plunger release mechanism is in the first state. The plunger release mechanism may be a mechanical lock which prevents movement of the plunger rod in the distal direction.

After the needle 5 is in the first position A and the needle shroud 11 is in the first needle shroud position C the drug delivery operation can be initialized by moving the housing 3 relative to the needle shroud 11. This movement advances the needle 5 in the distal direction so that the needle 5 pierces the skin 9. In addition, this movement causes the locking of the plunger release mechanism to be released. The plunger release mechanism releases the energy of the energy storage unit so that the drug is injected into the bulged area of the skin 9 located in the tissue receiving portion 8. This condition is shown in FIG. 3, there the needle 5 is in the second position B and the needle shroud 11 is in the second needle shroud position D.

After the drug delivery operation has been completed, the drug delivery arrangement may be removed from the skin 9. The needle shroud 11 may be biased relative to the housing 3. Thus, when the tissue receiving element 6 is removed from the skin 9 the needle shroud 11 is moved towards the first needle shroud position C. The needle shroud 11 can be moved distally, e.g. beyond the first needle shroud position C, into a final, locked position relative to the housing 3. In this position the needle shroud 11 is expediently axially locked relative to the housing 3 against movement in the proximal direction, e.g. by a locking engagement between a locking feature of the shroud 11 and the housing 3. As it is axially locked, the needle shroud 11 can no longer be displaced proximally relative to the housing 3. This protects the user from needle stick injuries after use.

The terms “drug” or “medicament” are used synonymously herein and describe a pharmaceutical formulation containing one or more active pharmaceutical ingredients or pharmaceutically acceptable salts or solvates thereof, and optionally a pharmaceutically acceptable carrier. An active pharmaceutical ingredient (“API”), in the broadest terms, is a chemical structure that has a biological effect on humans or animals. In pharmacology, a drug or medicament is used in the treatment, cure, prevention, or diagnosis of disease or used to otherwise enhance physical or mental well-being. A drug or medicament may be used for a limited duration, or on a regular basis for chronic disorders.

As described below, a drug or medicament can include at least one API, or combinations thereof, in various types of formulations, for the treatment of one or more diseases. Examples of API may include small molecules having a molecular weight of 500 Da or less; polypeptides, peptides and proteins (e.g., hormones, growth factors, antibodies, antibody fragments, and enzymes); carbohydrates and polysaccharides; and nucleic acids, double or single stranded DNA (including naked and cDNA), RNA, antisense nucleic acids such as antisense DNA and RNA, small interfering RNA (siRNA), ribozymes, genes, and oligonucleotides. Nucleic acids may be incorporated into molecular delivery systems such as vectors, plasmids, or liposomes. Mixtures of one or more drugs are also contemplated.

The drug or medicament may be contained in a primary package or “drug container” adapted for use with a drug delivery device. The drug container may be, e.g., a cartridge, syringe, reservoir, or other solid or flexible vessel configured to provide a suitable chamber for storage (e.g., short- or long-term storage) of one or more drugs. For example, in some instances, the chamber may be designed to store a drug for at least one day (e.g., 1 to at least 30 days). In some instances, the chamber may be designed to store a drug for about 1 month to about 2 years. Storage may occur at room temperature (e.g., about 20° C.), or refrigerated temperatures (e.g., from about-4° C. to about 4° C.). In some instances, the drug container may be or may include a dual-chamber cartridge configured to store two or more components of the pharmaceutical formulation to-be-administered (e.g., an API and a diluent, or two different drugs) separately, one in each chamber. In such instances, the two chambers of the dual-chamber cartridge may be configured to allow mixing between the two or more components prior to and/or during dispensing into the human or animal body. For example, the two chambers may be configured such that they are in fluid communication with each other (e.g., by way of a conduit between the two chambers) and allow mixing of the two components when desired by a user prior to dispensing. Alternatively or in addition, the two chambers may be configured to allow mixing as the components are being dispensed into the human or animal body.

The drugs or medicaments contained in the drug delivery devices as described herein can be used for the treatment and/or prophylaxis of many different types of medical disorders. Examples of disorders include, e.g., diabetes mellitus or complications associated with diabetes mellitus such as diabetic retinopathy, thromboembolism disorders such as deep vein or pulmonary thromboembolism. Further examples of disorders are acute coronary syndrome (ACS), angina, myocardial infarction, cancer, macular degeneration, inflammation, hay fever, atherosclerosis and/or rheumatoid arthritis. Examples of APIs and drugs are those as described in handbooks such as Rote Liste 2014, for example, without limitation, main groups 12 (anti-diabetic drugs) or 86 (oncology drugs), and Merck Index, 15th edition.

Examples of APIs for the treatment and/or prophylaxis of type 1 or type 2 diabetes mellitus or complications associated with type 1 or type 2 diabetes mellitus include an insulin, e.g., human insulin, or a human insulin analogue or derivative, a glucagon-like peptide (GLP-1), GLP-1 analogues or GLP-1 receptor agonists, or an analogue or derivative thereof, a dipeptidyl peptidase-4 (DPP4) inhibitor, or a pharmaceutically acceptable salt or solvate thereof, or any mixture thereof. As used herein, the terms “analogue” and “derivative” refers to a polypeptide which has a molecular structure which formally can be derived from the structure of a naturally occurring peptide, for example that of human insulin, by deleting and/or exchanging at least one amino acid residue occurring in the naturally occurring peptide and/or by adding at least one amino acid residue. The added and/or exchanged amino acid residue can either be codeable amino acid residues or other naturally occurring residues or purely synthetic amino acid residues. Insulin analogues are also referred to as “insulin receptor ligands”. In particular, the term “derivative” refers to a polypeptide which has a molecular structure which formally can be derived from the structure of a naturally occurring peptide, for example that of human insulin, in which one or more organic substituent (e.g. a fatty acid) is bound to one or more of the amino acids. Optionally, one or more amino acids occurring in the naturally occurring peptide may have been deleted and/or replaced by other amino acids, including non-codeable amino acids, or amino acids, including non-codeable, have been added to the naturally occurring peptide.

Examples of insulin analogues are Gly(A21), Arg(B31), Arg(B32) human insulin (insulin glargine); Lys(B3), Glu(B29) human insulin (insulin glulisine); Lys(B28), Pro(B29) human insulin (insulin lispro); Asp(B28) human insulin (insulin aspart); human insulin, wherein proline in position B28 is replaced by Asp, Lys, Leu, Val or Ala and wherein in position B29 Lys may be replaced by Pro; Ala(B26) human insulin; Des(B28-B30) human insulin; Des(B27) human insulin and Des(B30) human insulin.

Examples of insulin derivatives are, for example, B29-N-myristoyl-des(B30) human insulin, Lys(B29) (N-tetradecanoyl)-des(B30) human insulin (insulin detemir, Levemir®); B29-N-palmitoyl-des(B30) human insulin; B29-N-myristoyl human insulin; B29-N-palmitoyl human insulin; B28-N-myristoyl LysB28ProB29 human insulin; B28-N-palmitoyl-LysB28ProB29 human insulin; B30-N-myristoyl-ThrB29LysB30 human insulin; B30-N-palmitoyl-ThrB29LysB30 human insulin; B29-N—(N-palmitoyl-gamma-glutamyl)-des(B30) human insulin, B29-N-omega-carboxypentadecanoyl-gamma-L-glutamyl-des(B30) human insulin (insulin degludec, Tresiba®); B29-N—(N-lithocholyl-gamma-glutamyl)-des(B30) human insulin; B29-N—(ω-carboxyheptadecanoyl)-des(B30) human insulin and B29-N—(ω-carboxyheptadecanoyl) human insulin.

Examples of GLP-1, GLP-1 analogues and GLP-1 receptor agonists are, for example, Lixisenatide (Lyxumia®), Exenatide (Exendin-4, Byetta®, Bydureon®, a 39 amino acid peptide which is produced by the salivary glands of the Gila monster), Liraglutide (Victoza®), Semaglutide, Taspoglutide, Albiglutide (Syncria®), Dulaglutide (Trulicity®), rExendin-4, CJC-1134-PC, PB-1023, TTP-054, Langlenatide/HM-11260C (Efpeglenatide), HM-15211, CM-3, GLP-1 Eligen, ORMD-0901, NN-9423, NN-9709, NN-9924, NN-9926, NN-9927, Nodexen, Viador-GLP-1, CVX-096, ZYOG-1, ZYD-1, GSK-2374697, DA-3091, MAR-701, MAR709, ZP-2929, ZP-3022, ZP-DI-70, TT-401 (Pegapamodtide), BHM-034. MOD-6030, CAM-2036, DA-15864, ARI-2651, ARI-2255, Tirzepatide (LY3298176), Bamadutide (SAR425899), Exenatide-XTEN and Glucagon-Xten.

An example of an oligonucleotide is, for example: mipomersen sodium (Kynamro®), a cholesterol-reducing antisense therapeutic for the treatment of familial hypercholesterolemia or RG012 for the treatment of Alport syndrom.

Examples of DPP4 inhibitors are Linagliptin, Vildagliptin, Sitagliptin, Denagliptin, Saxagliptin, Berberine.

Examples of hormones include hypophysis hormones or hypothalamus hormones or regulatory active peptides and their antagonists, such as Gonadotropine (Follitropin, Lutropin, Choriongonadotropin, Menotropin), Somatropine (Somatropin), Desmopressin, Terlipressin, Gonadorelin, Triptorelin, Leuprorelin, Buserelin, Nafarelin, and Goserelin.

Examples of polysaccharides include a glucosaminoglycane, a hyaluronic acid, a heparin, a low molecular weight heparin or an ultra-low molecular weight heparin or a derivative thereof, or a sulphated polysaccharide, e.g. a poly-sulphated form of the above-mentioned polysaccharides, and/or a pharmaceutically acceptable salt thereof. An example of a pharmaceutically acceptable salt of a poly-sulphated low molecular weight heparin is enoxaparin sodium. An example of a hyaluronic acid derivative is Hylan G-F 20 (Synvisc®), a sodium hyaluronate.

The term “antibody”, as used herein, refers to an immunoglobulin molecule or an antigen-binding portion thereof. Examples of antigen-binding portions of immunoglobulin molecules include F(ab) and F(ab′)2 fragments, which retain the ability to bind antigen. The antibody can be polyclonal, monoclonal, recombinant, chimeric, de-immunized or humanized, fully human, non-human, (e.g., murine), or single chain antibody. In some embodiments, the antibody has effector function and can fix complement. In some embodiments, the antibody has reduced or no ability to bind an Fc receptor. For example, the antibody can be an isotype or subtype, an antibody fragment or mutant, which does not support binding to an Fc receptor, e.g., it has a mutagenized or deleted Fc receptor binding region. The term antibody also includes an antigen-binding molecule based on tetravalent bispecific tandem immunoglobulins (TBTI) and/or a dual variable region antibody-like binding protein having cross-over binding region orientation (CODV).

The terms “fragment” or “antibody fragment” refer to a polypeptide derived from an antibody polypeptide molecule (e.g., an antibody heavy and/or light chain polypeptide) that does not comprise a full-length antibody polypeptide, but that still comprises at least a portion of a full-length antibody polypeptide that is capable of binding to an antigen. Antibody fragments can comprise a cleaved portion of a full length antibody polypeptide, although the term is not limited to such cleaved fragments. Antibody fragments that are useful in the present invention include, for example, Fab fragments, F(ab′)2 fragments, scFv (single-chain Fv) fragments, linear antibodies, monospecific or multispecific antibody fragments such as bispecific, trispecific, tetraspecific and multispecific antibodies (e.g., diabodies, triabodies, tetrabodies), monovalent or multivalent antibody fragments such as bivalent, trivalent, tetravalent and multivalent antibodies, minibodies, chelating recombinant antibodies, tribodies or bibodies, intrabodies, nanobodies, small modular immunopharmaceuticals (SMIP), binding-domain immunoglobulin fusion proteins, camelized antibodies, and VHH containing antibodies. Additional examples of antigen-binding antibody fragments are known in the art.

The terms “Complementarity-determining region” or “CDR” refer to short polypeptide sequences within the variable region of both heavy and light chain polypeptides that are primarily responsible for mediating specific antigen recognition. The term “framework region” refers to amino acid sequences within the variable region of both heavy and light chain polypeptides that are not CDR sequences, and are primarily responsible for maintaining correct positioning of the CDR sequences to permit antigen binding. Although the framework regions themselves typically do not directly participate in antigen binding, as is known in the art, certain residues within the framework regions of certain antibodies can directly participate in antigen binding or can affect the ability of one or more amino acids in CDRs to interact with antigen.

Examples of antibodies are anti PCSK-9 mAb (e.g., Alirocumab), anti IL-6 mAb (e.g., Sarilumab), and anti IL-4 mAb (e.g., Dupilumab).

Pharmaceutically acceptable salts of any API described herein are also contemplated for use in a drug or medicament in a drug delivery device. Pharmaceutically acceptable salts are for example acid addition salts and basic salts.

Those of skill in the art will understand that modifications (additions and/or removals) of various components of the APIs, formulations, apparatuses, methods, systems and embodiments described herein may be made without departing from the full scope and spirit of the present invention, which encompass such modifications and any and all equivalents thereof.

An example drug delivery device may involve a needle-based injection system as described in Table 1 of section 5.2 of ISO 11608-1:2014(E). As described in ISO 11608-1:2014(E), needle-based injection systems may be broadly distinguished into multi-dose container systems and single-dose (with partial or full evacuation) container systems. The container may be a replaceable container or an integrated non-replaceable container.

As further described in ISO 11608-1:2014(E), a multi-dose container system may involve a needle-based injection device with a replaceable container. In such a system, each container holds multiple doses, the size of which may be fixed or variable (pre-set by the user). Another multi-dose container system may involve a needle-based injection device with an integrated non-replaceable container. In such a system, each container holds multiple doses, the size of which may be fixed or variable (pre-set by the user).

As further described in ISO 11608-1:2014(E), a single-dose container system may involve a needle-based injection device with a replaceable container. In one example for such a system, each container holds a single dose, whereby the entire deliverable volume is expelled (full evacuation). In a further example, each container holds a single dose, whereby a portion of the deliverable volume is expelled (partial evacuation). As also described in ISO 11608-1:2014(E), a single-dose container system may involve a needle-based injection device with an integrated non-replaceable container. In one example for such a system, each container holds a single dose, whereby the entire deliverable volume is expelled (full evacuation). In a further example, each container holds a single dose, whereby a portion of the deliverable volume is expelled (partial evacuation).

### REFERENCE NUMERALS

drug delivery arrangement 1
drug delivery device 2
housing 3
drug container 4
needle 5
tissue receiving element 6
needle passage opening 7
tissue receiving portion 8
skin 9
wall 10
wall inner surface 10.1
arm 10.2
needle shroud 11
cap 12
cap recess 12.1
needle shield 13
first position needle A
second position needle B
first position needle shroud C
second position needle shroud D

1. A drug delivery arrangement (1), comprising:
a drug delivery device (2) having a housing (3), the housing being provided to receive a drug container (4) and a needle (5), and
a tissue receiving element (6) having a needle passage opening (7) and a tissue receiving portion (8), wherein
the tissue receiving element is arranged to contact a skin (9) of a patient during use of the drug delivery arrangement, wherein
the tissue receiving portion extends in the distal direction away from the needle passage opening, wherein
the drug delivery arrangement is configured such that the needle passage opening and the needle are movable relative to one another from a first position (A), in which the needle is arranged outside of the tissue receiving portion, into a second position (B), in which the needle is arranged within the tissue receiving portion, wherein
the needle enters the tissue receiving portion during the relative movement from the first position into the second position to penetrate tissue arranged in the tissue receiving portion, wherein
the tissue receiving element is configured to interact with the skin in order to fill the tissue receiving portion with tissue, and wherein the tissue receiving element is part of or attachable to the drug delivery device.

2. The drug delivery arrangement (1) according to claim 1, wherein
the tissue receiving portion (8) has a larger diameter in a distal end region than the needle passage opening (7).

3. The drug delivery arrangement (1) according to claim 1 or 2, wherein
the tissue receiving element (6) comprises a wall (10) extending in the distal direction as seen from the needle passage opening (7), and wherein
the tissue receiving portion (8) is laterally delimited by an inner surface (10.1) of the wall.

4. The drug delivery arrangement (1) according to claim 3, wherein
the tissue receiving element (6) comprises arms (10.2) defining the wall (10), and wherein
the arms are diametrically opposed in the radial direction of the drug delivery arrangement.

5. The drug delivery arrangement (1) according to any of the claims 3 and 4, wherein
the wall (10) has a U-shaped cross section.

6. The drug delivery arrangement (1) according to any of the claims 3 to 5, wherein
the drug delivery arrangement comprises a needle shroud (11) with a needle shroud body, and wherein
the tissue receiving element (6) is arranged at the distal end of the needle shroud body or attachable to the distal end of the needle shroud body.

7. The drug delivery arrangement (1) according to claim 6, wherein
the tissue receiving element (6) is detachably connected to the needle shroud body.

8. The drug delivery arrangement (1) according to any of the claims 3 to 5, wherein
the tissue receiving element (6) is the distal end portion of the housing (3).

9. The drug delivery arrangement (1) according to any one of the preceding claims, wherein
the drug delivery arrangement further comprises:
an energy storage unit having energy stored therein for driving a plunger in the distal direction relative to the housing (3) to dispense a dose of drug from the drug container (4), and
a plunger release mechanism configured to have a first state and a second state, and wherein
the stored energy of the energy storage unit is released when the plunger release mechanism is in the second state.

10. The drug delivery arrangement (1) according to claim 9, wherein
the drug delivery device (2) is configured such that the plunger release mechanism is switched from the first state to the second state after the needle (5) is moved relative to the needle passage opening (7) from the first position (A) to the second position (B).

11. The drug delivery arrangement (1) according to any of the claim 6 or 7 in combination with any of the claims 9 and 10, wherein
the needle shroud (11) is axially movable relative to the housing (3) from a first needle shroud position (C) to a second needle shroud position (D), and wherein
the plunger release mechanism is configured such that the plunger release mechanism is switched from the first state to the second state after the needle shroud (11) is moved from the first needle shroud position (C) to the second needle shroud position (D).

12. The drug delivery arrangement (1) according to any of the claim 6 or 7 in combination with any of the claims 9 to 11, wherein
the needle (5) is fixed relative to the housing (3).

13. The drug delivery arrangement (1) according to any of the claims 1 to 5 and 8 to 10, wherein
the needle (5) is movable relative to the housing (3).

14. The drug delivery arrangement (1) according to any one of the preceding claims, wherein
the drug delivery device (2) is an auto-injector.

15. The drug delivery arrangement (1) according to any one of the preceding claims, wherein
the drug delivery arrangement comprises a cap (12), and wherein
the cap comprises recesses in the radial direction which are suitable for receiving a portion of the tissue receiving element (6).

16. The drug delivery arrangement (1) according to any one of the preceding claims, wherein the drug container (4) is a syringe,

17. The drug delivery arrangement (1) according to any one of the preceding claims, wherein
the drug delivery arrangement comprises a cap (12), and wherein
the cap (12) comprises a needle shield remover.

18. The drug delivery arrangement (1) according to any one of the preceding claims, wherein
the tissue receiving portion (8) tapers continuously as seen from its distal end towards the needle passage opening (7).

19. A drug delivery arrangement (1), comprising:
a drug delivery device (2) having a housing (3), the housing being provided to receive a drug container (4) and a needle (5), and
a tissue receiving element (6) having a needle passage opening (7) and a tissue receiving portion (8), wherein
the tissue receiving element is arranged to contact a skin (9) of a patient during use of the drug delivery arrangement, wherein
the tissue receiving portion extends in the distal direction away from the needle passage opening, wherein
the drug delivery arrangement is configured such that the needle passage opening and the needle are movable relative to one another from a first position (A), in which the needle is arranged outside of the tissue receiving portion, into a second position (B), in which the needle is arranged within the tissue receiving portion, wherein
the needle enters the tissue receiving portion during the relative movement from the first position into the second position to penetrate tissue arranged in the tissue receiving portion, wherein
the tissue receiving element is configured to interact with the skin in order to fill the tissue receiving portion with tissue, wherein
the tissue receiving element is part of or attachable to the drug delivery device, wherein
the tissue receiving element comprises a wall (10) extending in the distal direction as seen from the needle passage opening, wherein
the tissue receiving portion is laterally delimited by an inner surface (10.1) of the wall, wherein
the drug delivery arrangement comprises a needle shroud (11) with a needle shroud body, and wherein
the tissue receiving element is attachable to the distal end of the needle shroud body.
